(19) United States
(12) Patent Application Publication
(10) Pub. No.: US 2012/0317001 A1
(43) Pub. Date: Dec. 13, 2012

(54) IMPLANT INVENTORY MANAGEMENT SYSTEM

(75) Inventors: Brian C. DeBusk, Knoxville, TN (US); Angela M. Sewell, Knoxville, TN (US); John G. Jacobs, Knoxville, TN (US); Rex A. Hurd, Knoxville, TN (US); Elizabeth A. White, Strawberry Plains, TN (US); Jeffrey D. Griffith, Knoxville, TN (US); Samuel J. E. Fox, Oliver Springs, TN (US)

(73) Assignee: DEROYAL INDUSTRIES, INC., Powell, TN (US)

(21) Appl. No.: 13/154,880

(22) Filed: Jun. 7, 2011

(51) Int. Cl. G06Q 10/00

(52) U.S. Cl. 705/29; 705/28

(57) ABSTRACT

An inventory management system manages information related to items, such as implants, used during a medical surgical procedure. The inventory management system includes an inventory management computer and an item inventory database. The inventory management computer executes an inventory management application to process item usage information. The item usage information includes item identification information and quantity information for each item used during performance of the medical surgical procedure. The inventory management application associates the item usage information with a particular surgical case at a particular customer surgical facility. The inventory management computer receives the item usage information from a customer computer at the customer surgical facility via a wide area network, such as the Internet. The item inventory database, which is in communication with the inventory management computer, stores item inventory information reflecting the inventory of items at the customer surgical facility.

### FIELD

This invention relates to an inventory management system. More particularly, this invention relates to a system for managing inventories of items packaged in kits for use in surgical procedures involving implants.

### BACKGROUND

Items to be consumed during medical surgical procedures are often provided in kits that are customized for the particular surgical procedure to be performed. For an implant procedure, these kits include various sizes of the implant, screws for securing the implant, and tools to be used during the procedure. After completion of the procedure, the kits must be replenished to replace items that were consumed. In the past, the replenishment was usually performed by a sales representative of the company that manufactured the kit. For example, the sales representative had a locker in a supply room in the hospital where the representative kept an inventory of items to be used to replace the consumed items. In most circumstances, the sales representative had to manually keep records of items consumed and the quantities of items to be ordered to replace inventory extracted from the locker.

Unfortunately, the inventory replenishment procedure described above was fraught with recording errors and outright fraud, particularly in situations where the sales representative collected a commission based on the number of items consumed during a surgical procedure.

What is needed, therefore, is a system for recording usage of consumable surgical items, ordering replacement items, receiving the items into inventory, and maintaining accurate records of consumed, ordered, and received items.

### SUMMARY

The above and other needs are met by an inventory management system for managing information related to items, such as implants, used during a medical surgical procedure. In one embodiment, the inventory management system includes an inventory management computer and an item inventory database. The inventory management computer executes an inventory management application to process item usage information which includes item identification information and quantity information for each item used during performance of the medical surgical procedure. The inventory management application associates the item usage information with a particular surgical case at a particular customer surgical facility. The inventory management computer is in communication with a wide area network, such as the Internet, through which the inventory management application receives the item usage information from a customer computer associated with the customer surgical facility. The item inventory database, which is in communication with the inventory management computer, stores item inventory information.

In some embodiments, the item usage information also includes information indicating whether an item was implanted during the surgical procedure or was wasted.

In some embodiments, the inventory management application is configured to communicate a listing of sets of items through the wide area network for display on the customer computer. The inventory management application of these embodiments is also configured to receive a selection from the customer computer indicating which of the sets of items in the listing is to be associated with the particular surgical case, and to generate an inventory control document for the selected set of items on which a user at the customer surgical facility may record the item usage information.

In some embodiments, the inventory management application is configured to communicate a listing of surgical cases associated with the customer surgical facility through the wide area network for display on the customer computer, and to receive a selection from the customer computer indicating a surgical case in the listing for which item usage is to be recorded. For the selected surgical case, the inventory management application receives usage information entered by a user on the customer computer identifying a set of items used during the surgical procedure, identifying each item in the set that was used during the surgical procedure, indicating a quantity of each item used, and indicating whether each item used was implanted during the surgical procedure or was wasted. The inventory management application then updates the item inventory information in the item inventory database based on the usage information received from the customer computer.

In some embodiments, the inventory management application receives usage information that is manually entered using an input device connected to the customer computer. In other embodiments, the inventory management application receives usage information entered using a barcode reader connected to the customer computer.

In some embodiments, the inventory management system includes an admissions-discharge-transfer (ADT) computer system associated with the customer surgical facility. The ADT computer system is configured to receive and process case information for the particular surgical case, and is in communication with the inventory management computer via the wide area network. The inventory management application of these embodiments is configured to receive the case information from the ADT computer system and associate the case information with the item usage information for the particular surgical case.

In some embodiments, the inventory management application communicates a listing of sets of items in inventory at the customer surgical facility through the wide area network for display on the customer computer. The inventory management application receives a selection through the wide area network from the customer computer indicating a set of items in the listing that the user desires to replenish with items from inventory at the customer surgical facility. The inventory management application also generates an inventory picking document for the selected set of items which the user at the customer surgical facility can use as a guide in replenishing the selected set of items with items from inventory at the customer surgical facility.

In some embodiments, the item inventory database maintains a record of items in inventory at the customer surgical facility which are available to replenish sets of items depleted during a surgical procedure at the customer surgical facility. The inventory management application of these embodiments communicates a listing of sets of items through the wide area network for display on the customer computer, wherein the listing indicates sets of items recorded in the item inventory database as being in inventory at the customer surgical facility. The inventory management application receives a selection through the wide area network from the customer computer indicating a set of items that are to be transferred out of the inventory at the customer surgical facility because one or more of the items have been used to replenish a set of items depleted during a surgical procedure. The inventory management application also generates a usage list for the selected set of items which is displayed on the customer computer. The user at the customer surgical facility may enter quantities of items on the usage list to be transferred from inventory at the customer facility to reflect actual usage of the items during the surgical procedure. The inventory management application operates on the item inventory database to adjust a record of items in inventory at the customer surgical facility to reflect actual usage as indicated by quantities of items entered by the user on the usage list.

In another aspect, the invention provides a computer-implemented method for managing information related to implants used during a medical surgical procedure at a customer surgical facility. The method includes receiving implant usage information at an inventory management computer via a wide area network from a customer computer associated with the customer surgical facility. The implant usage information includes implant identification information and implant quantity information for each implant used during performance of the medical surgical procedure. The method also includes associating the implant usage information with a particular surgical case at the customer surgical facility in an implant inventory database.

### BRIEF DESCRIPTION OF THE DRAWINGS

Further advantages of the invention are apparent by reference to the detailed description in conjunction with the figures, wherein elements are not to scale so as to more clearly show the details, wherein like reference numbers indicate like elements throughout the several views, and wherein:

FIG. 1 is a functional block diagram of an embodiment of an implant inventory management system;

FIGS. 2-17 depict flowcharts describing the operation of embodiments of an implant inventory management system;

FIGS. 18 and 19 depict interface display screens as viewable by a user during operation of embodiments an implant inventory management system;

FIG. 20 depicts an inventory control form used during operation of embodiments of an implant inventory management system;

FIGS. 21-41 depict interface display screens as viewable by a user during operation of embodiments an implant inventory management system; and

FIG. 42 depicts an embodiment of a tray of implants for use in a medical procedure.

### DETAILED DESCRIPTION

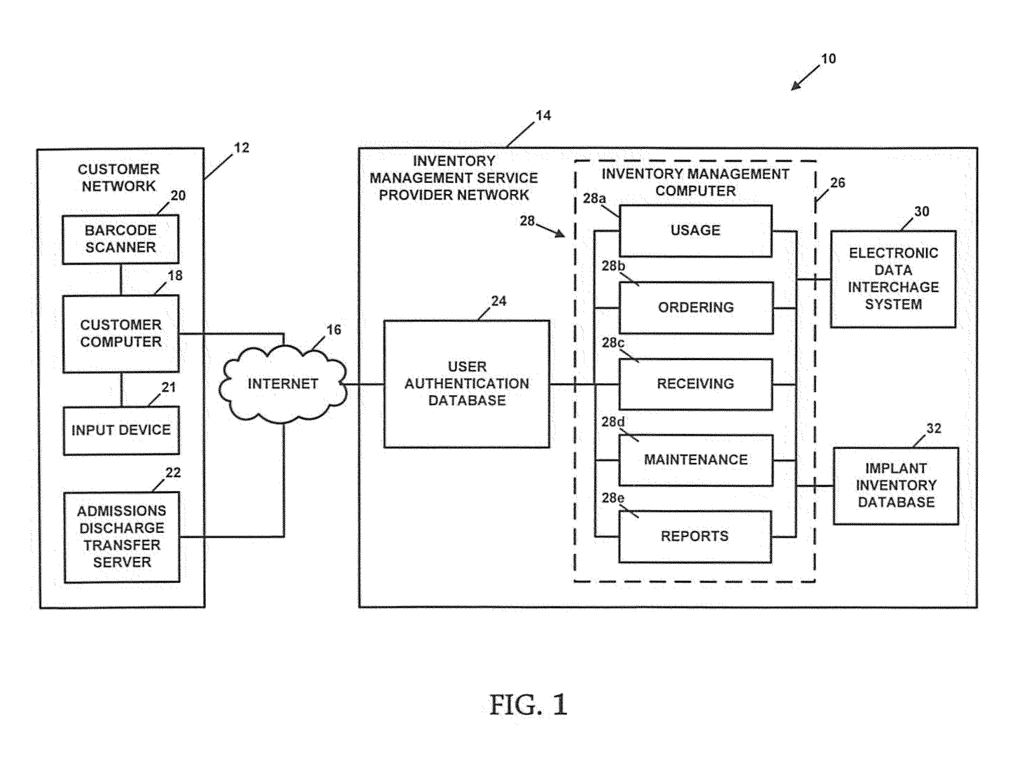

FIG. 1 depicts an embodiment of an implant inventory management system 10. As the term is used herein, an “implant” is a medical device or piece of material, such as a plate or screw, which may be used in a medical surgical procedure to repair or replace part of the body. As described in more detail hereinafter, the implant inventory management system 10 provides computer-implemented tools and processes for managing an inventory of medical implants for use in medical procedures. However, one skilled in the art will recognize that the implant inventory management system 10 may be used to manage inventories of other components and materials in medical and nonmedical applications. Thus, the inventions described herein are not limited to implant inventory applications or medical component inventory applications.

As shown in FIG. 1, the implant inventory management system 10 includes a customer computer network 12 and an inventory management service provider computer network 14 which communicate with each other via a communication network such as the Internet 16. As the term is used herein, “customer” generally refers to a medical facility where surgeries or other medical procedures are performed, such as a hospital or outpatient surgical center, which would consume medical implants during the surgeries or other procedures. The term “customer” may also refer to any consumer of goods that are inventoried using the inventory management system described herein. Accordingly, one or more of the components of the customer computer network 12 may be located within a hospital or other medical facility.

The customer computer network 12 includes one or more customer computers 18 that are operable to communicate through the Internet 16 with the inventory management service provider computer network 14. The customer computer 18 may be, for example, a desktop computer, laptop computer, tablet computer, or smart phone. A scanning device 20, such as a barcode scanner or RFID reader, is connected to the customer computer 18, and the customer computer 18 includes software needed to interface and operate with the scanning device 20. A user input device 21, such as a keyboard or mouse or both, is also connected to the customer computer 18.

The customer computer network 12 also includes an admissions-discharge-transfer (ADT) server 22. The ADT server 22 comprises one or more computers that store and manage records regarding the status of patients receiving treatment in a medical facility. Generally, a patient's status is either admitted to the facility, discharged from the facility, or transferred to another facility.

With continued reference to FIG. 1, the inventory management service provider computer network 14 comprises one or more computers which store information and execute software for managing inventories of materials and devices, such as medical implants, tools, and other materials used by the customer in performing medical procedures. As the term is used herein, an “inventory management service provider” may be a company that maintains inventories of implants and other medical devices that are supplied to the customer for use during the performance of medical procedures. An example of one such service provider is DeRoyal Industries, Inc. of Powell, Tenn. Alternatively, the “inventory management service provider” may not maintain the inventory, but may provide inventory management services to another company that does maintain the inventory.

In the embodiment of FIG. 1, the inventory management service provider computer network 14 includes a user authentication database 24, an inventory management computer 26 running an inventory management application 28, an electronic data interchange server 30, and an implant inventory database 32. The user authentication database 24 stores records for each customer/user of the of the inventory management service provider, such as user ID's, passwords, and billing information. The electronic data interchange server 30, which may be a J.D. Edwards/Oracle server, executes programs for implementing electronic commerce transactions between the inventory management service provider network 14 and the customer network 12. The implant inventory database 32 stores records indicating quantities of each type of implant in inventory.

The inventory management application 28 running on the inventory management computer 26 includes a Usage Module 28a, an Ordering Module 28b, a Receiving Module 28c, a Maintenance Module 28d, and a Reports Module 28e. The functionality of each of these modules is described in more detail hereinafter.

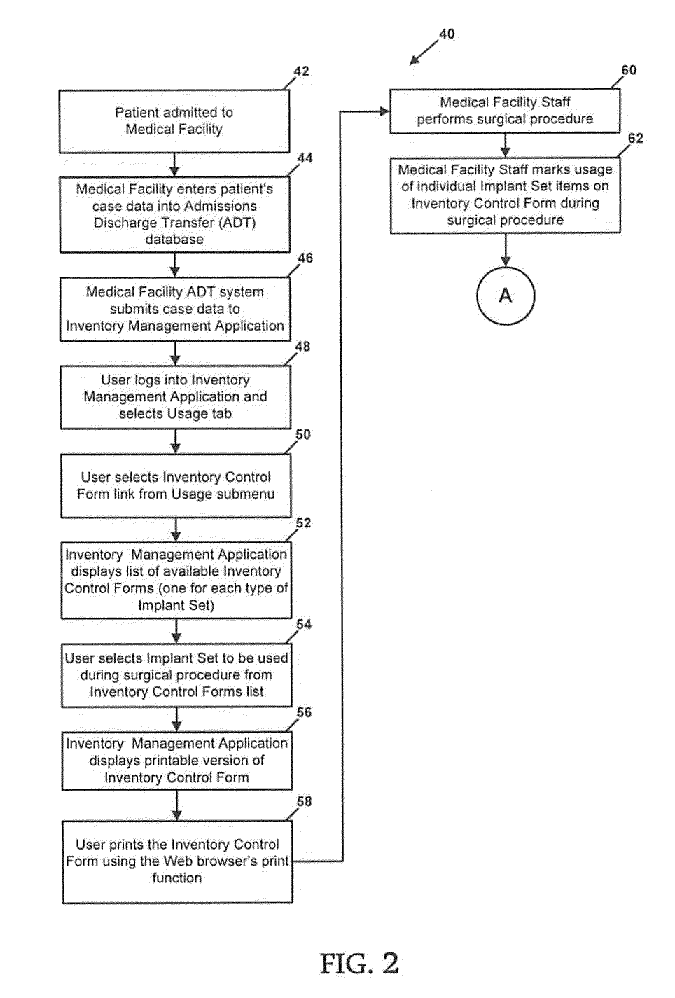

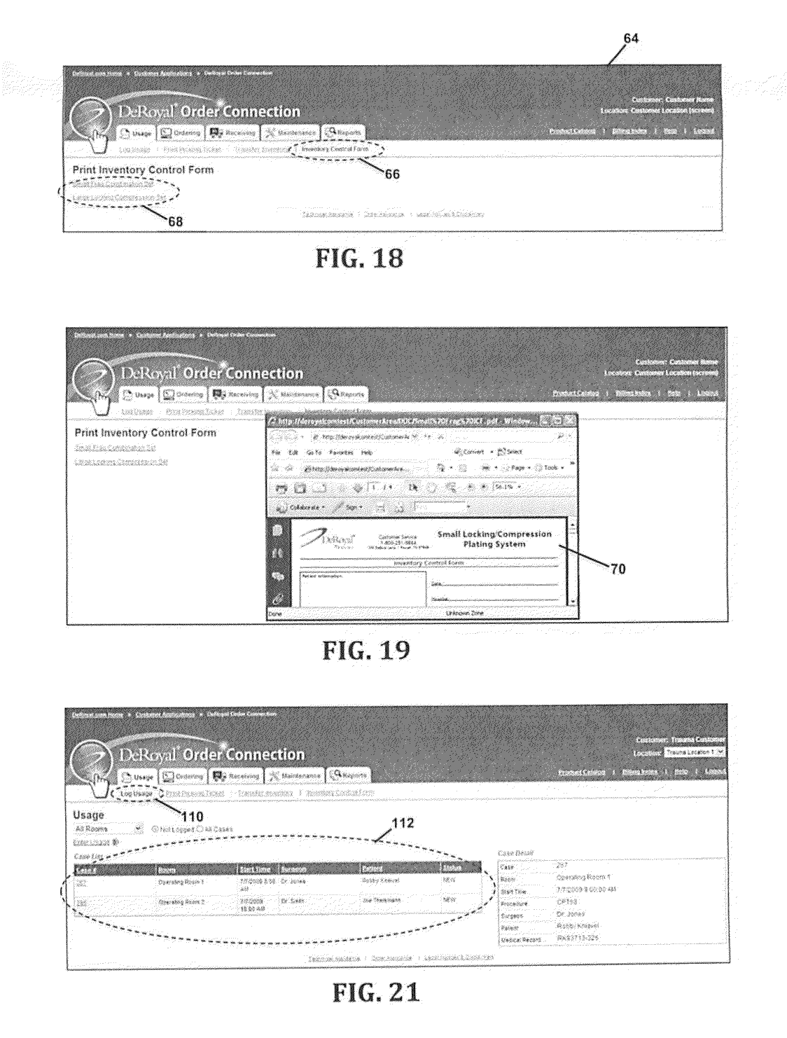

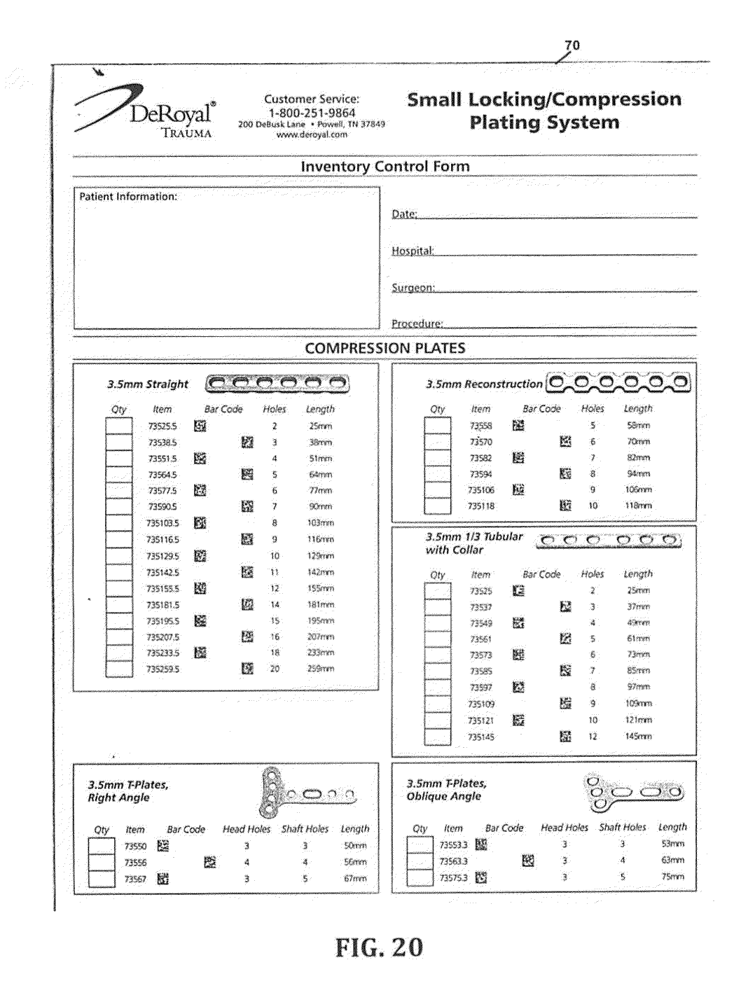

FIG. 2 depicts a process 40 for logging implant usage during a surgical procedure using an Inventory Control Form generated by the Usage Module 28a. When a surgical patient is admitted to the medical facility (step 42), medical facility personnel enter the patient's case data into the ADT server 22 (step 44). The case data is also submitted to the inventory management application 28 running on the inventory management computer 26 (step 46). Using a browser application running on the client computer 18, a user logs into the inventory management application 28 running on the inventory management computer 26 via the Internet 16 and selects the Usage tab on the browser screen 64 as depicted in FIG. 18 (step 48). This initiates execution of the Usage Module 28a. On the Usage screen, the user selects the Inventory Control Form link 66 (step 50) which causes a list 68 of available Inventory Control Forms to be displayed (step 52). Preferably, one Inventory Control Form is displayed for each type of available implant set. In the example of FIG. 18, two implant sets are available: “Small Frag Combination Set” and “Large Locking Compression Set.” When the user selects the implant set to be used in the surgical procedure from the list 68 (step 54), a printable version of the associated Inventory Control Form 70 is displayed (step 56) as shown in FIG. 19. The user then can print a copy of the Inventory Control Form 70, an example of which is shown in FIG. 20, to use during the surgical procedure (step 58). Preferably, during performance of the surgical procedure (step 60), a member of the staff logs the usage of individual implant set items on the form 70 (step 62). Alternatively, the staff member logs the usage on the form 70 after completion of the procedure.

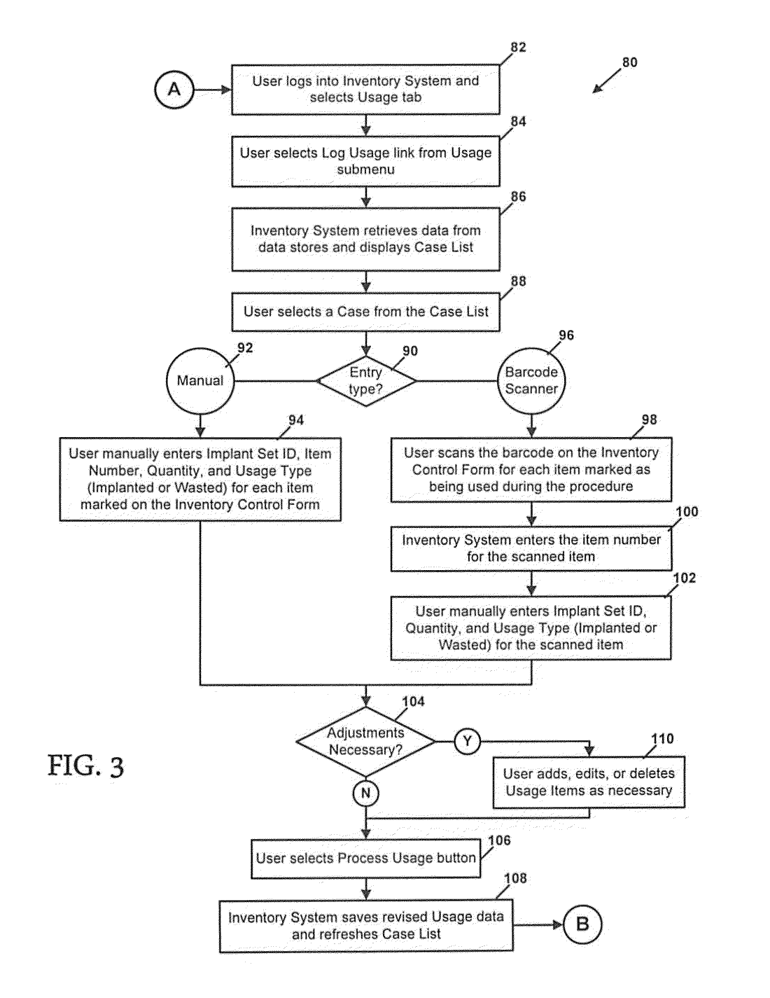

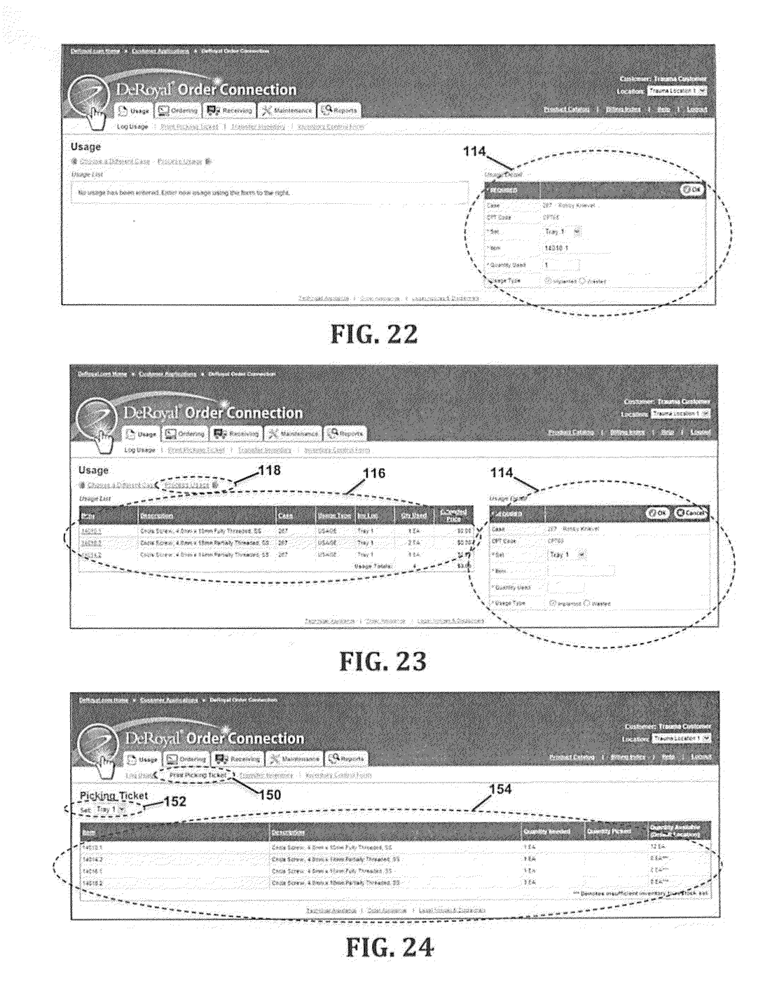

FIG. 3 depicts a process 80 for logging implant usage recorded on the Inventory Control Form 70 into the implant inventory database 32. To begin this process, a user logs into the inventory management application 28 running on the inventory management computer 26 via the Internet 16 and selects the Usage tab on the browser screen as depicted in FIG. 21 (step 82). This initiates execution of the Usage Module 28a. In the Usage submenu, the user selects the Log Usage link 110 (step 84) which causes the retrieval of case data from the implant inventory database 32 (step 86). The case data is displayed in a case list 112 from which the user selects a case under which implant usage will be logged (step 88). For example, if the user selects case number 267 from the case list 112, and no usage has yet been logged for this case, the screen as depicted in FIG. 22 will be displayed. For manual logging, the user keys in usage information in the Usage Detail block 114 using the input device 21. The usage information preferably includes the implant set ID number (such as Tray 1), an item number (such as 14010.1), quantity used (such as 1), and usage type (Implanted or Wasted) for each item marked on the Inventory Control Form (step 94). If logging using a barcode scanner, the user scans the barcode on the Inventory Control Form for each item marked as being used in the procedure (step 98), and the inventory management application 28 automatically enters the item number for the scanned item into the Usage block 114 (step 100). The user then manually enters the rest of the usage information in the Usage Detail block 114 (step 102). FIG. 23 depicts a list of items 116 entered as used for case number 267. If any adjustments are needed (step 104), the user may add, edit, or delete usage items by clicking on the item in the list 116 and making the changes in the input block 114 (step 110). When satisfied that the usage is correctly entered, the user clicks on the Process Usage link 118 (step 106) at which point the inventory management application 28 saves the revised usage data to the implant inventory database 32 and refreshes the case list 112 (step 108).

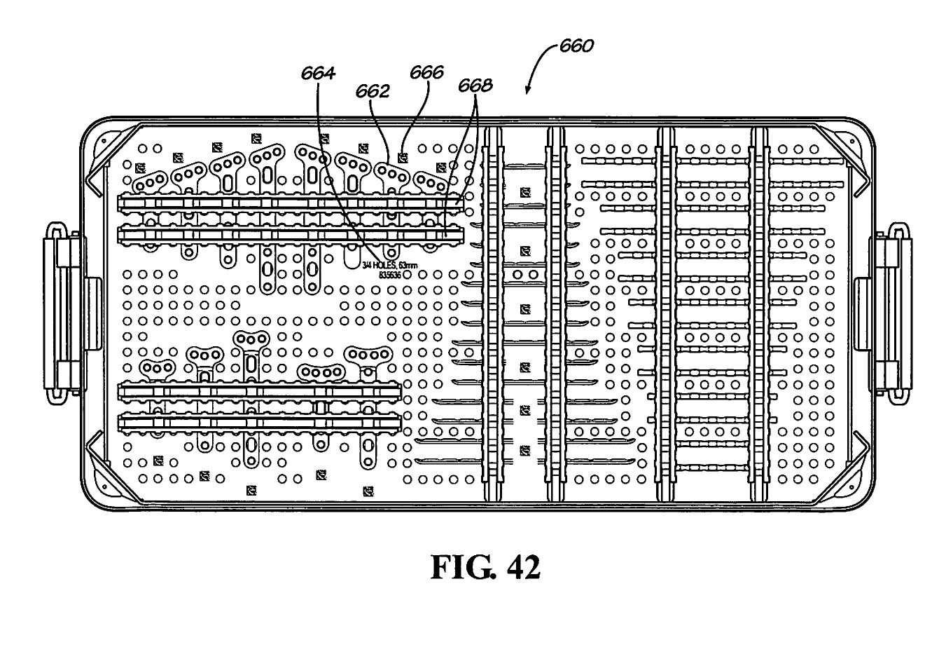

In an alternative embodiment, instead of using an inventory control form 70 to record implant and material usage to be logged, a user gathers this information by visually inspecting an implant kit tray that was used in a medical procedure. An example of such a tray 660 is depicted in FIG. 42. Each component on the tray is held in place in slots in a rack 668. A designated location for each component is indicated by a printed outline 662 of the component, a description with item number 664, and a barcode 666. If a component is not present in its designated slot after a procedure, it is considered to have been used during the procedure. A user may log usage by manually entering the item number 664 for the used component using the input device 21 as described above (step 94 of FIG. 3) or by scanning the barcode 666 as described above (step 100 of FIG. 3).

In another alternative embodiment, the user gathers the usage information by taking a digital photograph of the implant kit tray 660 that was used in a medical procedure. This photograph is emailed or otherwise electronically communicated to the inventory management computer 26 where the Usage Module 28a or other component of the inventory management application 28 automatically detects empty slots in the item racks 668, reads the barcodes 666 associated with those empty slots, and logs usage of the items in the implant inventory database 32.

In yet another alternative embodiment, the inventory management application 28 displays a graphic of the tray 660 on the browser of the customer computer 18. The graphic includes “radio buttons” or other graphic indicators (such as check boxes) next to the outline 662 of the used components. The user indicates usage of items by pressing the radio button (or entering a check in a check box) next to the used items, and the inventory management application 28 logs usage of the items in the implant inventory database 32.

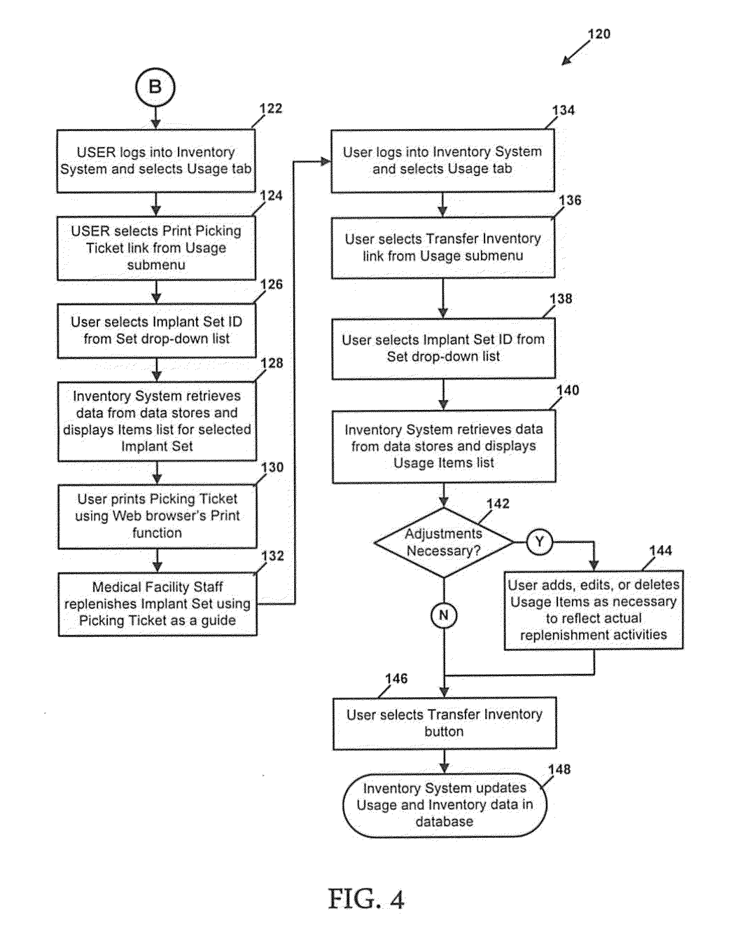

FIG. 4 depicts a process 120 for replenishing an implant set tray with implants, tools, or other materials that the customer used during a medical procedure. To begin this process, a user logs into the inventory management application 28 running on the inventory management computer 26 via the Internet 16 and selects the Usage tab on the browser screen as depicted in FIG. 24 (step 122). In the Usage submenu, the user selects the Print Picking Ticket link 150 which causes the drop-down list 152 of implant set IDs to appear (step 124). The user then selects one of the implant set IDs from the list 152 (step 126) at which point the inventory management application 28 retrieves data from the implant inventory database 32 for the selected implant set and displays the item list 154 for the selected set on the screen (step 128). Using the browser's print function, the user can now print the Picking Ticket (step 130). In preferred embodiments, the Picking Ticket shows all items that have been used and provides a list of items and the quantity of each to be retrieved from the supply closet to replenish the implant set tray. The Picking Ticket will also indicate the quantity of items available in the supply closet, and whether there is sufficient inventory available there to restock the tray. Using the Picking Ticket as a guide, the medical facility staff can replenish the implant set tray using the available stock in the supply closet (step 132).

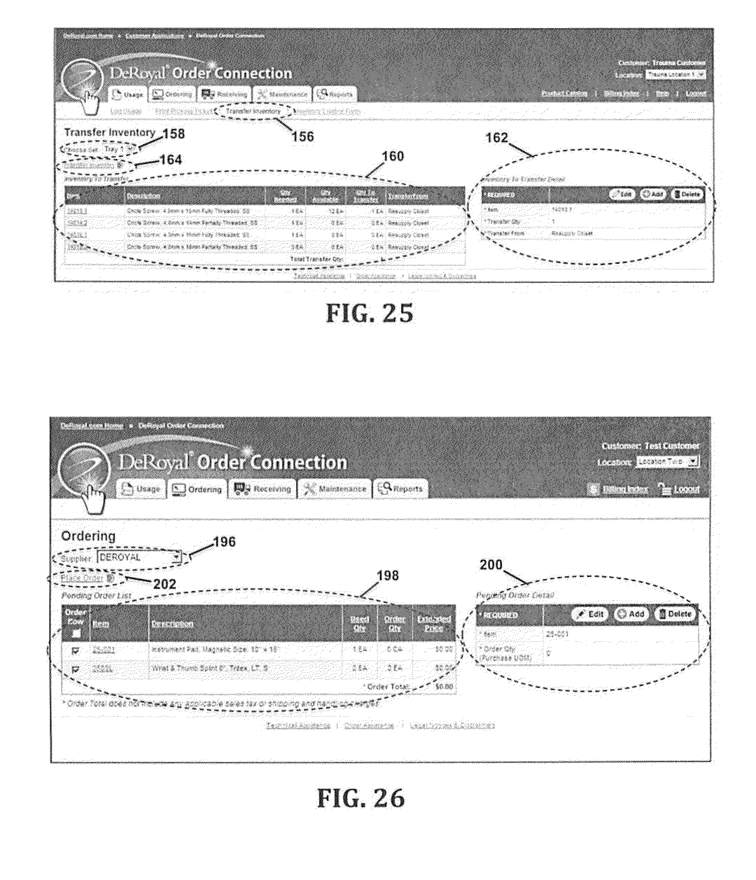

With continued reference to FIG. 4, a user can record the transfer of inventory from the supply closet to the implant tray. To begin this process, a user logs into the inventory management application 28 running on the inventory management computer 26 via the Internet 16 and selects the Usage tab on the browser screen as depicted in FIG. 25 (step 134). In the Usage submenu, the user selects the Transfer Inventory link 156 which causes the drop-down list 158 of implant set IDs to appear (step 136). The user then selects one of the implant set IDs from the list 158 (step 138) at which point the inventory management application 28 retrieves data from the implant inventory database 32 for the selected implant set and displays on the screen the list 160 of items to transfer from inventory for the selected set (step 140). If any adjustments are needed (step 142), the user may add, edit, or delete usage items by clicking on the item in the list 160 and making the changes in the input block 162 (step 144). When satisfied that the transfer information is correctly entered, the user clicks on the Transfer Inventory link 164 (step 146) at which point the inventory management application 28 updates the usage and inventory data in the implant inventory database 32 (step 148).

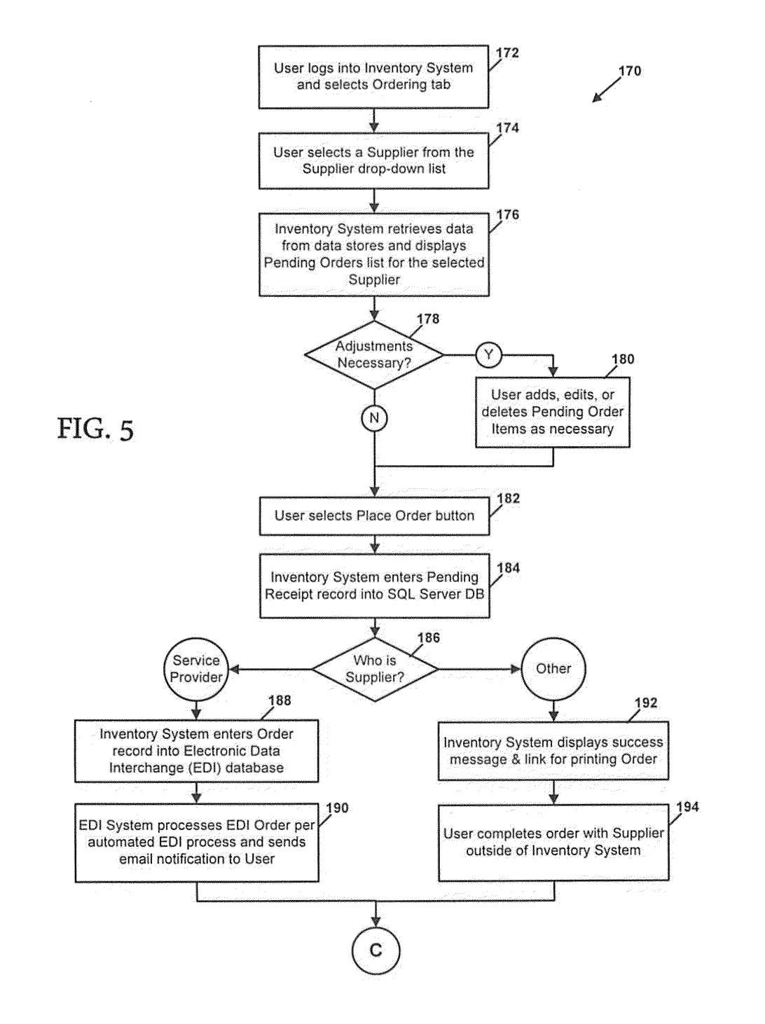

FIG. 5 depicts a process 170 for ordering items from a supplier to replenish stock in the customer supply closet. To begin this process, a user logs into the inventory management application 28 running on the inventory management computer 26 via the Internet 16 and selects the Ordering tab on the browser screen as depicted in FIG. 26 (step 172). The user then selects a Supplier from the supplier drop-down list 196 (step 174). The inventory management application 28 retrieves data indicating pending orders for items and displays this information in a list 198 on the screen (step 176). This list 198 shows the number of items that have been used since the last order was placed with the supplier. The list also shows the quantity of each item to be ordered and the price of each item. If adjustments to the list 198 are needed (step 178), the user can add, edit, or delete items from the pending order using the input block 200. When satisfied that the list is complete, the user selects the Place Order link 202 (step 182) at which point the inventory management application 28 enters a pending receipt record in the implant inventory database 32 (step 184). If the supplier selected at step 174 is also the inventory management service provider, the inventory management application 28 enters the order record into the EDI system 30 (step 188), and the EDI system 30 processes the EDI order record according to a standardized EDI process and sends a confirmatory email to the user (step 190). If the supplier selected at step 174 is not the inventory management service provider, the inventory management application 28 displays a success message and a link for printing an order form the supplier (step 192). The user then completes the order using the order form outside the implant inventory management system 10 (step 194).

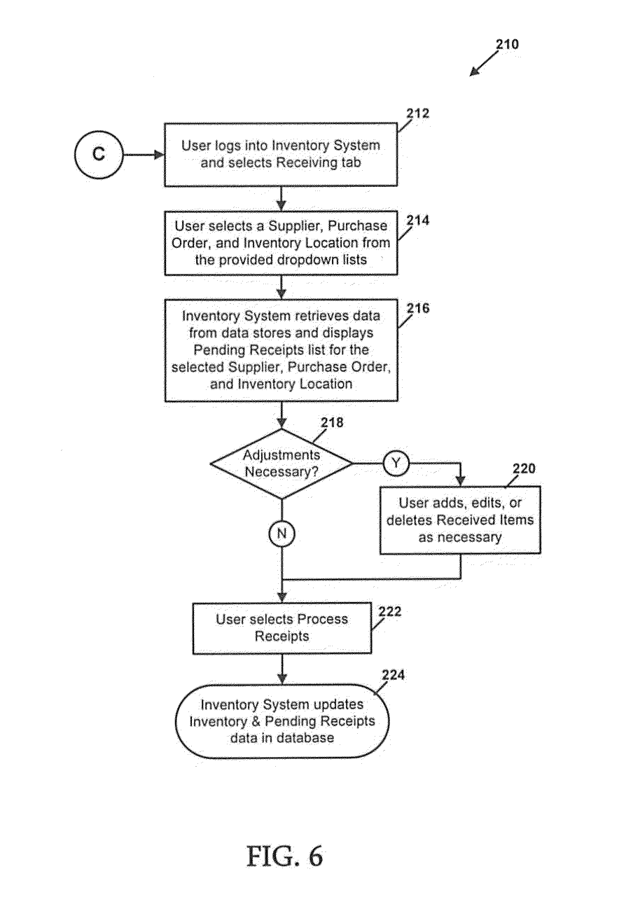

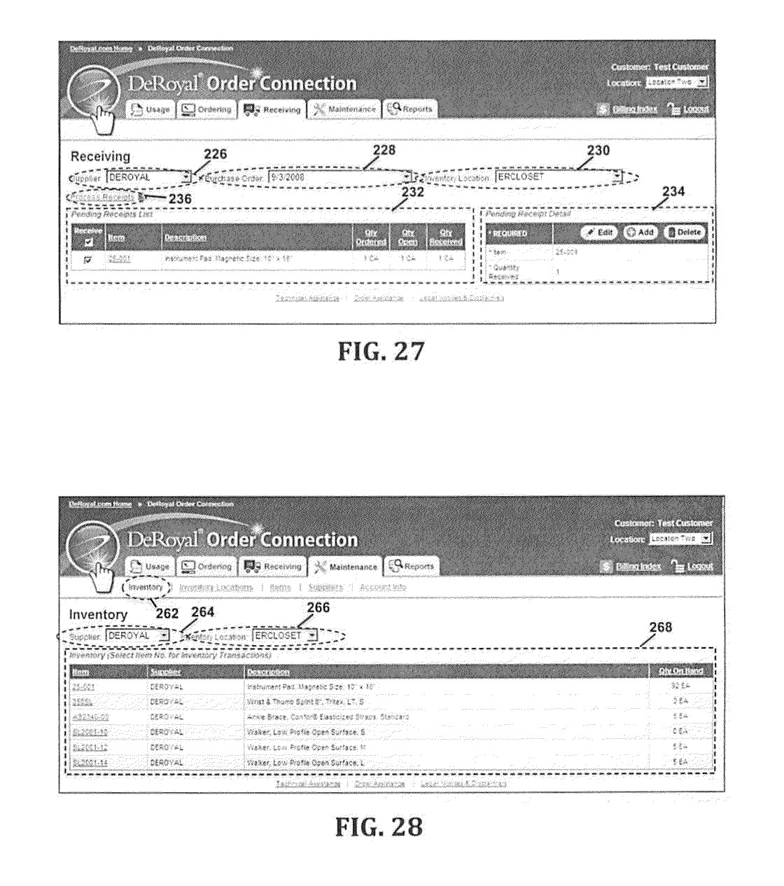

FIG. 6 depicts a process 210 for receiving items ordered from a supplier into customer inventory. To begin this process, a user logs into the inventory management application 28 running on the inventory management computer 26 via the Internet 16 and selects the Receiving tab on the browser screen as depicted in FIG. 27 (step 212). The user then selects a Supplier from the supplier drop-down list 226, a purchase order from drop-down list 228, and an inventory location from drop-down list 230 (step 214). The inventory management application 28 retrieves data indicating pending receipts for items for the selected supplier and purchase order, and displays this information in a list 232 on the screen (step 216). This list 232 shows items that the customer has ordered and should be receiving. The selected inventory location determines to which location the system will add the received items. In the example of FIG. 27, the selected inventory location is “ERCLOSET.” The inventory location is important, because in preferred embodiments, the system will not allow use of more inventory than is available in the inventory location. As shown in FIG. 27, for each item, the list 232 includes the item number, description, quantity ordered, quantity open, and quantity received. If the customer received the products listed, the user clicks on the box under “Receive” and the quantity received field will be populated with the quantity ordered. If adjustments in the quantity received are needed, the user may use the input block 234 to add, edit, or delete received items (step 220). When modifications are complete, and all items to be received have the check mark, the user selects the Process Receipts link 236 (step 222) at which point the inventory management application 28 updates the inventory and pending receipts in the implant inventory database 32 (step 224).

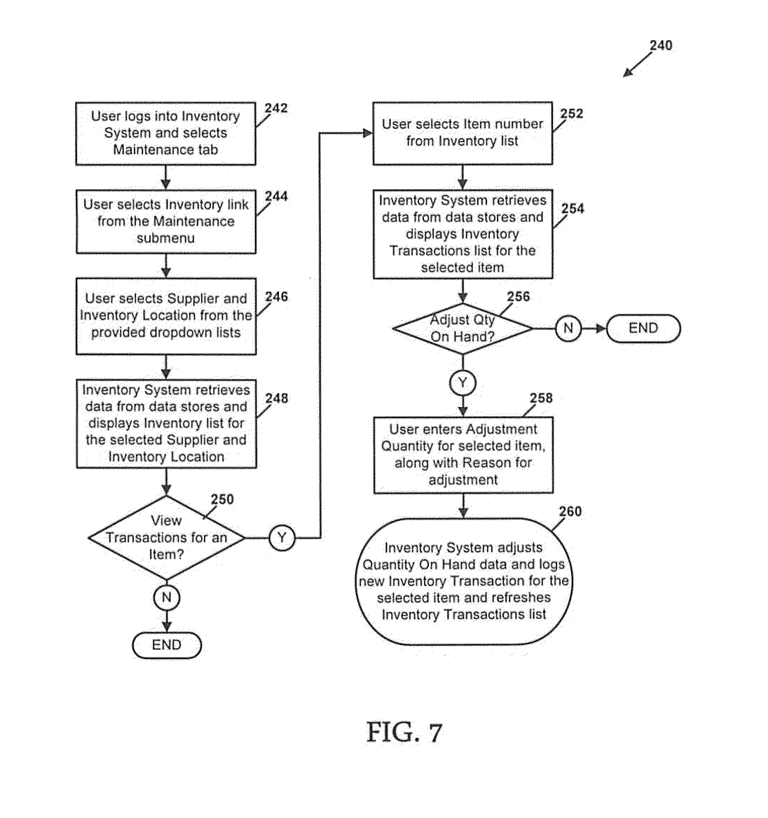

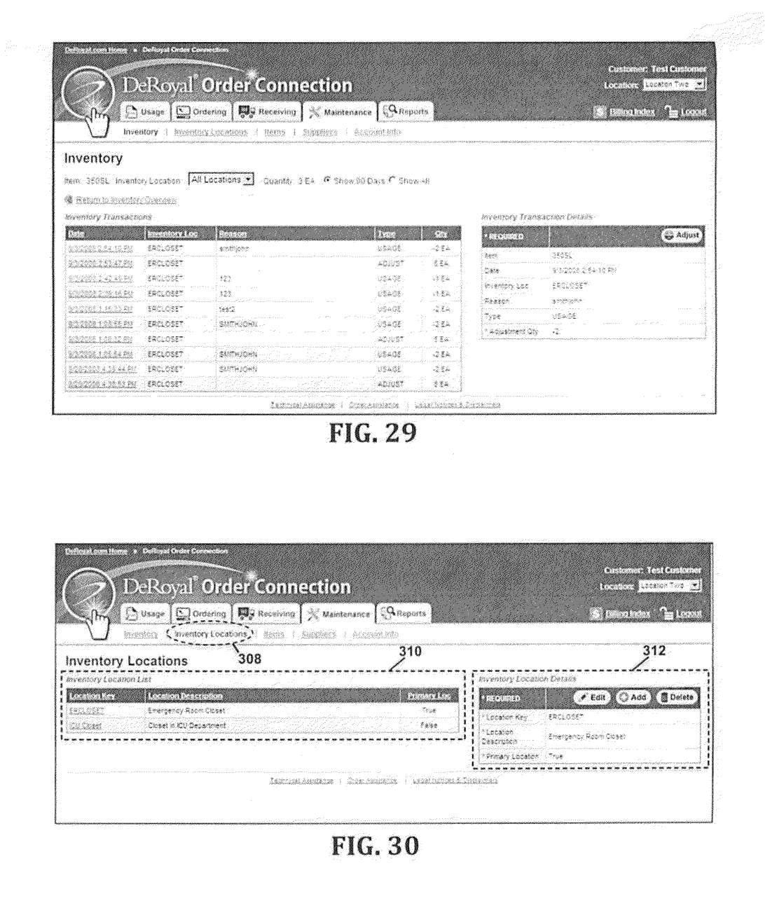

FIG. 7 depicts a process 240 for viewing and maintaining a customer's inventory. To begin this process, a user logs into the inventory management application 28 running on the inventory management computer 26 via the Internet 16 and selects the Maintenance tab on the browser screen as depicted in FIG. 28 (step 242) and selects the Inventory link 262 in the Maintenance submenu (step 244). The user then selects a Supplier from the supplier drop-down list 264 and an inventory location from drop-down list 266 (step 246). The inventory management application 28 retrieves data indicating inventory from the selected supplier and inventory location, and displays this information in a list 268 on the screen (step 248). If a user wants to view transactions for an item, the user selects the item from the list 268 (step 252), and the system retrieves and displays inventory data for the selected item (step 254) as shown in FIG. 29. The user can then adjust the quantity on hand for the selected item and enter a reason for the adjustment (step 258). The inventory management application 28 then adjusts the quantity on hand accordingly, logs the inventory transaction, and refreshes the inventory transactions list (step 260).

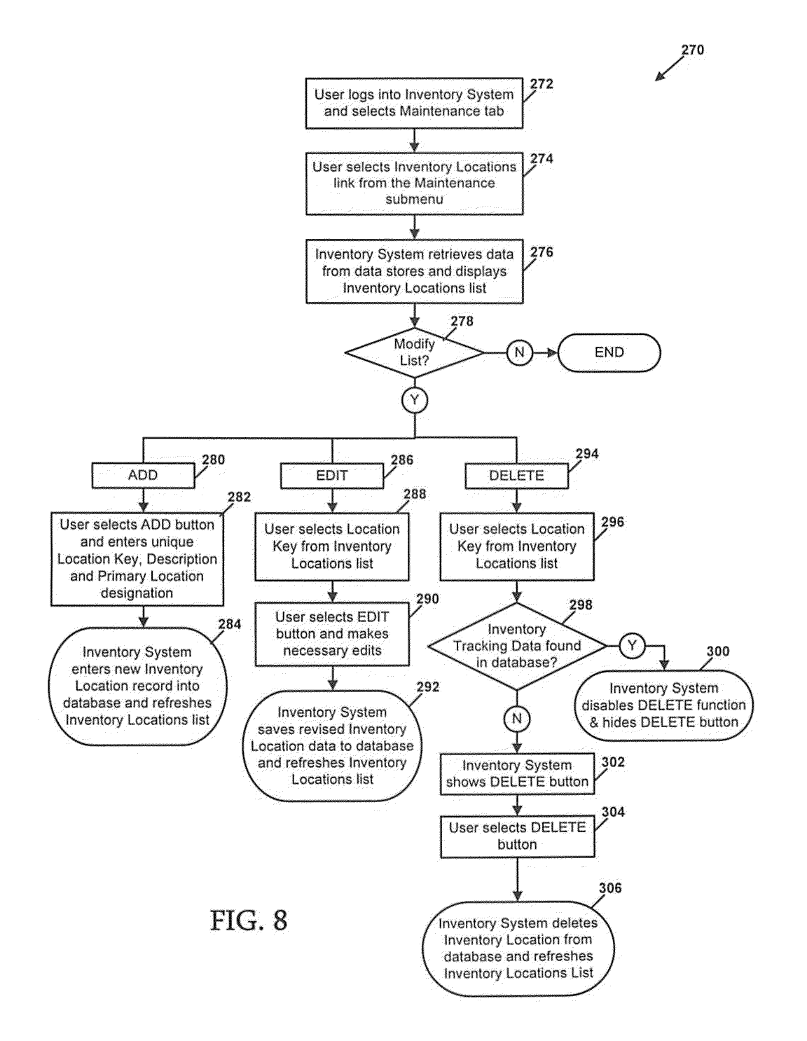

FIG. 8 depicts a process 270 for adding and deleting a customer's inventory locations, and changing the customer's primary location at which inventory is stored. To begin this process, a user logs into the inventory management application 28 running on the inventory management computer 26 via the Internet 16 and selects the Maintenance tab on the browser screen as depicted in FIG. 30 (step 272) and selects the Inventory Locations link 308 in the Maintenance submenu (step 274). The inventory management application 28 retrieves data indicating the customer's inventory locations, and displays this information in an inventory location list 310 on the screen (step 276). Using the options provided in the Inventory Location Details box 312, the user can add locations (steps 280-284), edit locations (steps 286-292), or delete locations (steps 294-306) as shown in FIG. 8.

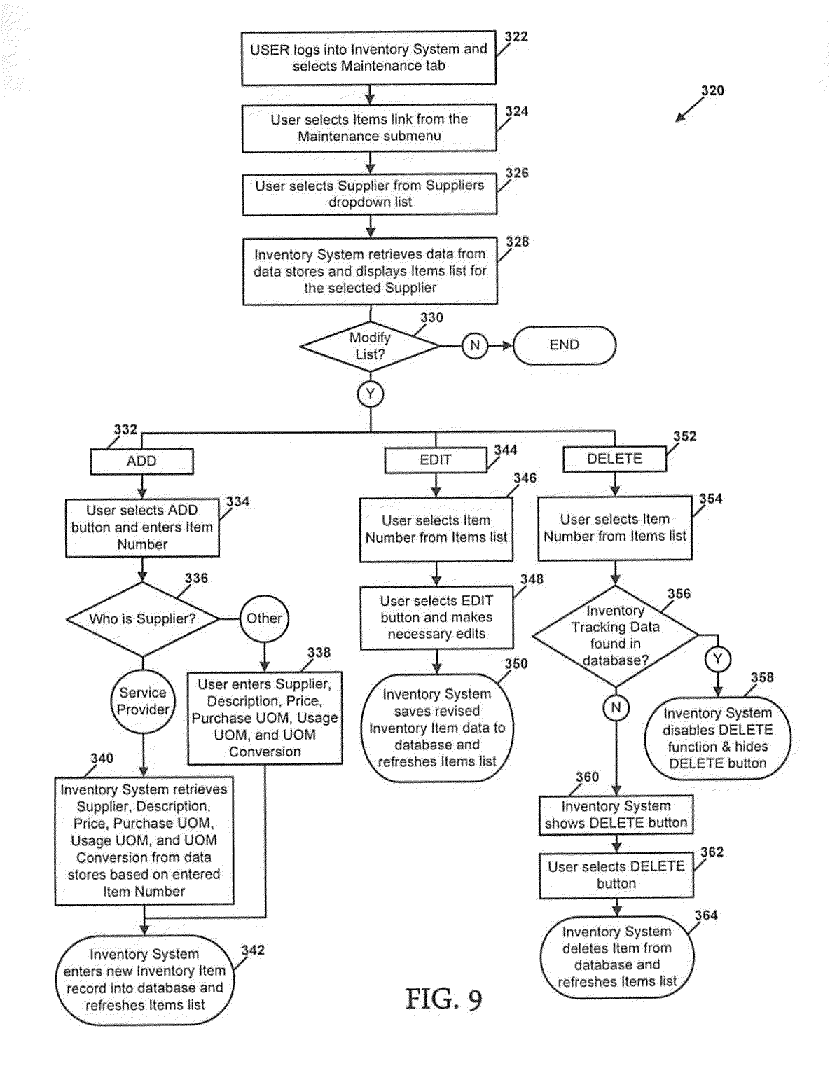

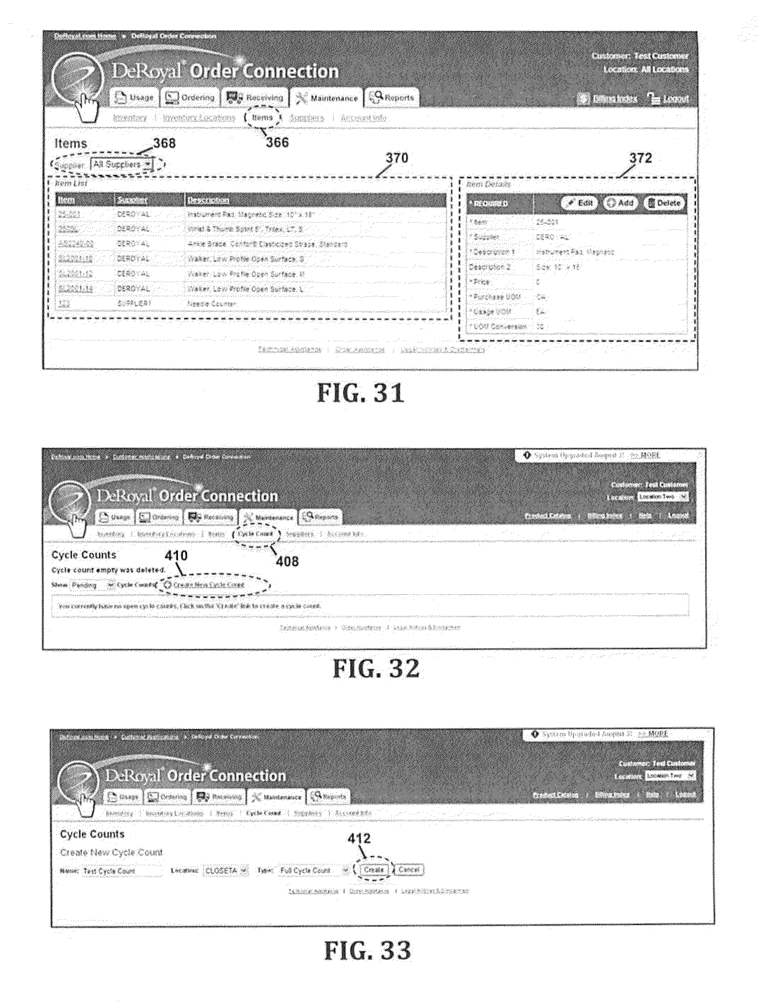

FIG. 9 depicts a process 320 which allows the customer to add items to inventory that the customer wishes to begin ordering from a supplier. To begin this process, a user logs into the inventory management application 28 running on the inventory management computer 26 via the Internet 16 and selects the Maintenance tab on the browser screen as depicted in FIG. 31 (step 322) and selects the Items link 366 in the Maintenance submenu (step 324). The user then selects a particular supplier or “all suppliers” from a supplier drop-down list 368 (step 326). The inventory management application 28 retrieves data indicating the items available from the selected supplier(s), and displays this information in an items list 370 on the screen (step 328). Using the options provided in the Item Details box 372, the user can add items (steps 332-342), edit items (steps 344-350), or delete locations (steps 352-364) as shown in FIG. 9.

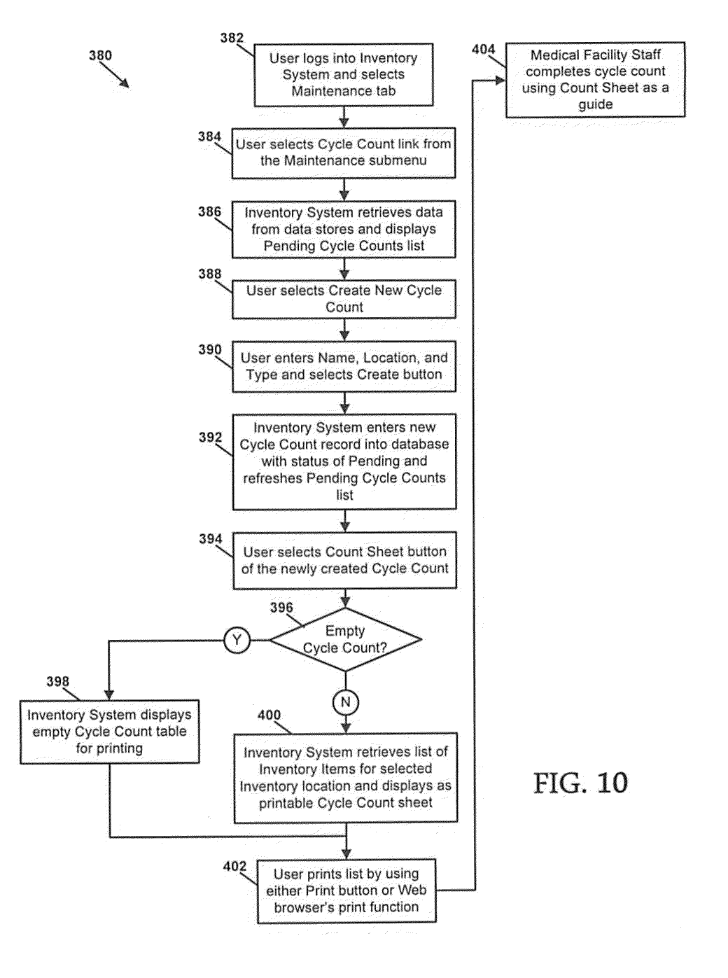

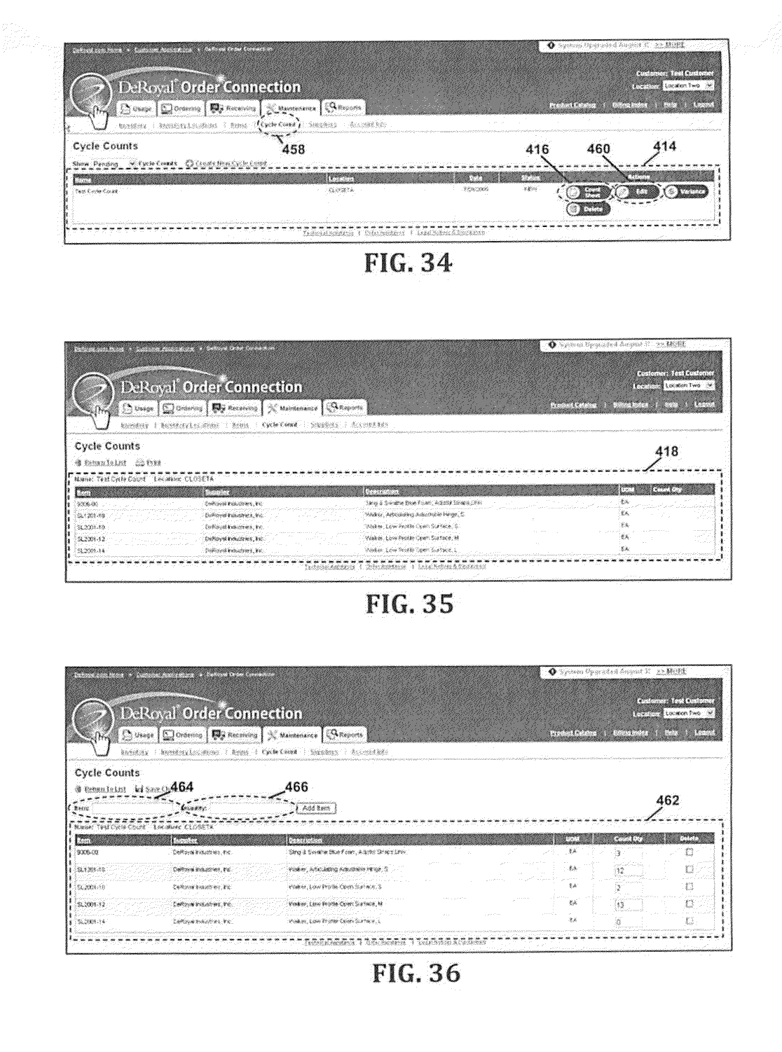

FIG. 10 depicts a process 380 which allows the customer to create and view cycle counts for the items in inventory, where a cycle count is a count of a subset of a total inventory which is taken at some particular frequency or cycle. To begin this process, a user logs into the inventory management application 28 running on the inventory management computer 26 via the Internet 16 and selects the Maintenance tab on the browser screen as depicted in FIG. 32 (step 382) and selects the Cycle Count link 408 in the Maintenance submenu (step 384). The inventory management application 28 retrieves cycle count data and displays this information as a Pending Cycle Counts list on the screen (step 386). If no open cycle counts exist, a corresponding message is displayed as shown in FIG. 32. The user then selects the Create New Cycle Count link 410 (step 388), at which point data entry dialog boxes are displayed which allow for entry of a cycle count name, inventory location, and type of cycle count (step 390). Upon selection of the Create button 412, the inventory management application 28 enters a new pending cycle count record into the implant inventory database 32 and refreshes the Pending Cycle Counts list 414 as shown in FIG. 34 (step 392). For the newly created cycle count, the user selects the Count Sheet button 416 (step 394). If the cycle count is empty (step 396), the inventory management application 28 displays an empty cycle count table for printing (step 398). If the cycle count is empty (step 396), the inventory management application 28 retrieves a list of inventory items for the selected inventory location and displays the list 418 as a cycle count sheet (step 400) that the user may print (step 402) as shown in FIG. 35. The user may then complete the cycle count using the cycle count sheet as a guide (step 404).

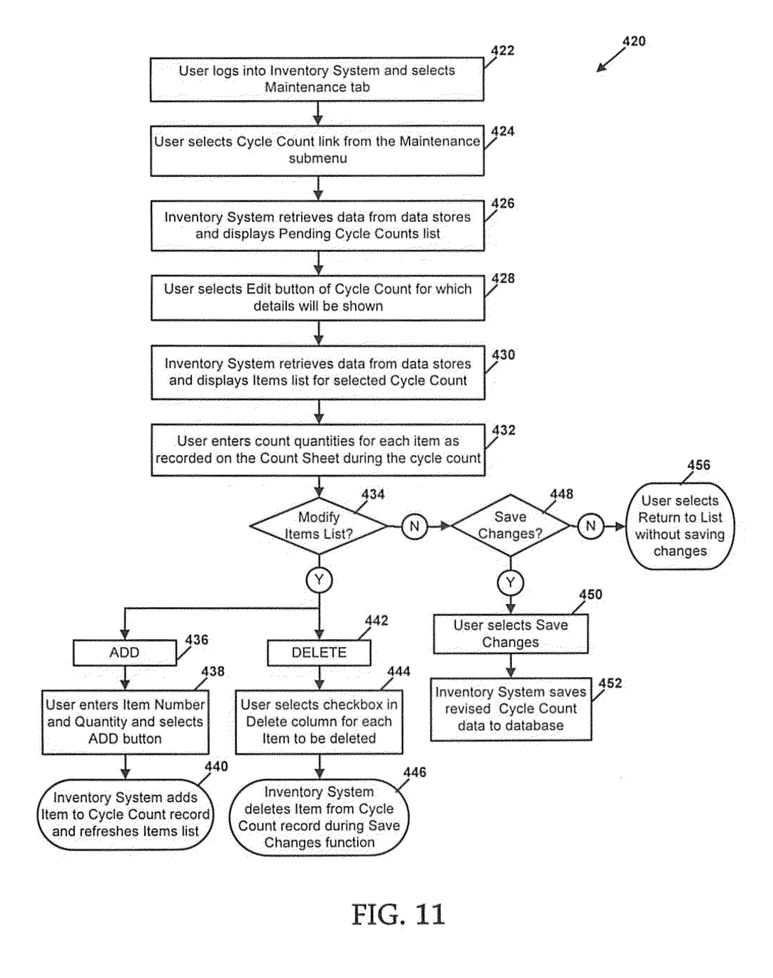

FIG. 11 depicts a process 420 which allows the customer to edit a cycle count. To begin this process, a user logs into the inventory management application 28 running on the inventory management computer 26 via the Internet 16 and selects the Maintenance tab on the browser screen as depicted in FIG. 34 (step 422) and selects the Cycle Count link 458 in the Maintenance submenu (step 424). The inventory management application 28 retrieves cycle count data and displays this information as a Pending Cycle Counts list 414 on the screen (step 426). The user clicks on the Edit button 460 of the cycle count to be edited (step 428) at which point the inventory management application 28 retrieves item data for the selected cycle count and displays this information as an Items list 462 on the screen as shown in FIG. 36 (step 430). The user can then enter count quantities for each item as recorded on the Count Sheet (step 432) using the Item box 464 and the Quantity box 466 as set forth in steps 436-440. The user can also delete items (steps 442-446), and then save the changes (steps 448-456) as shown in FIG. 11.

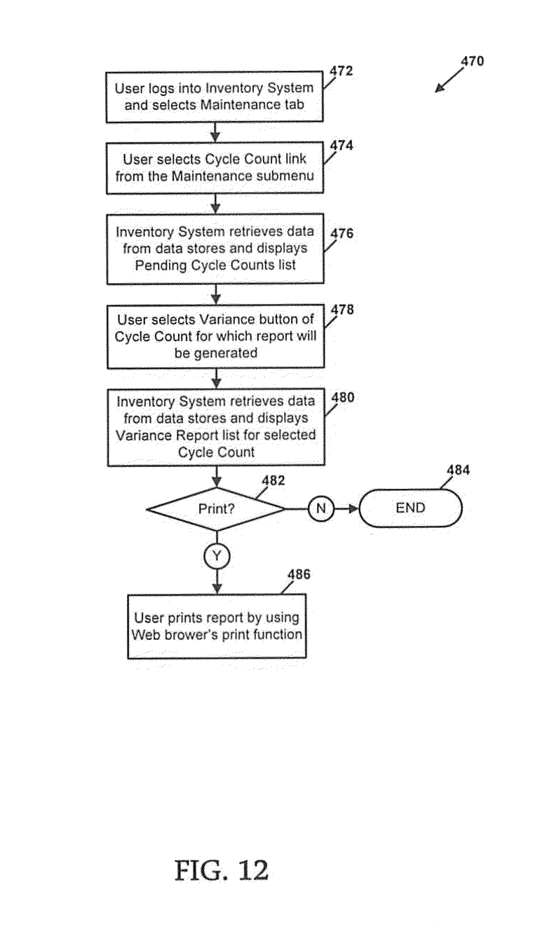

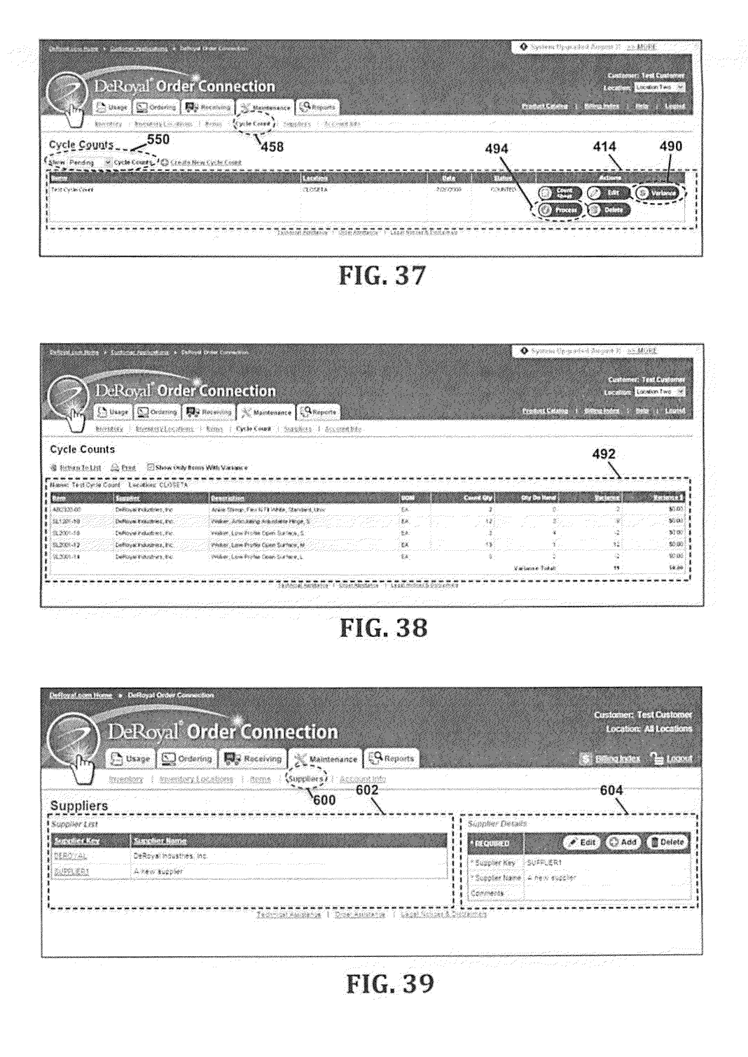

FIG. 12 depicts a process 470 which allows the customer to view and print a variance report. To begin this process, a user logs into the inventory management application 28 running on the inventory management computer 26 via the Internet 16 and selects the Maintenance tab on the browser screen as depicted in FIG. 37 (step 472) and selects the Cycle Count link 458 in the Maintenance submenu (step 474). The inventory management application 28 retrieves cycle count data and displays this information as a Pending Cycle Counts list 414 on the screen (step 476). The user clicks on the Variance button 490 of the cycle count for which a variance report is desired (step 478) at which point the inventory management application 28 retrieves variance data for the selected cycle count and displays this information as a Variance Report 492 on the screen as shown in FIG. 38 (step 480). The Variance Report lists quantities and dollar differences between items on hand and the number of items counted. The user can print the report such as by using the browser's print function (steps 482 and 486).

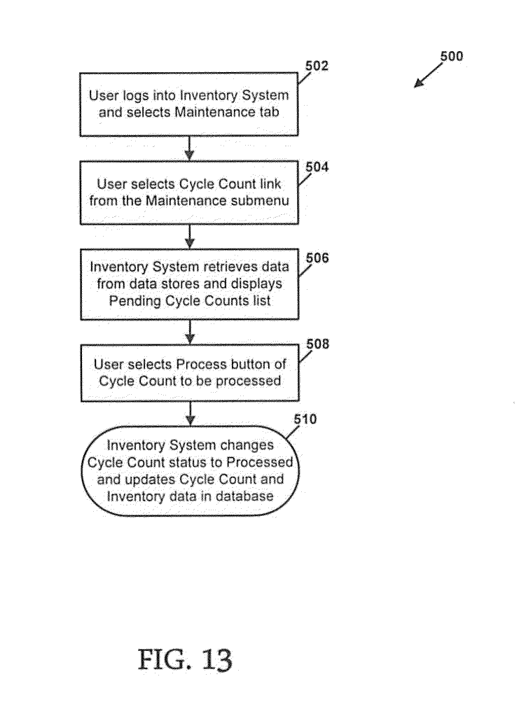

FIG. 13 depicts a process 500 which allows the customer to process the cycle count once every item in the cycle count has a count quantity. The Process function makes all necessary adjustments to the quantity on hand to reflect the quantity actually counted in the cycle count. To begin this process, a user logs into the inventory management application 28 running on the inventory management computer 26 via the Internet 16 and selects the Maintenance tab on the browser screen as depicted in FIG. 37 (step 502) and selects the Cycle Count link 458 in the Maintenance submenu (step 504). The inventory management application 28 retrieves cycle count data and displays this information as a Pending Cycle Counts list 414 on the screen (step 506). The user clicks on the Process button 494 of the cycle count to be processed (step 508) at which point the inventory management application 28 processes and updates the cycle count and the implant inventory database 32 and changes the cycle count status to reflect that it has been processed (step 510).

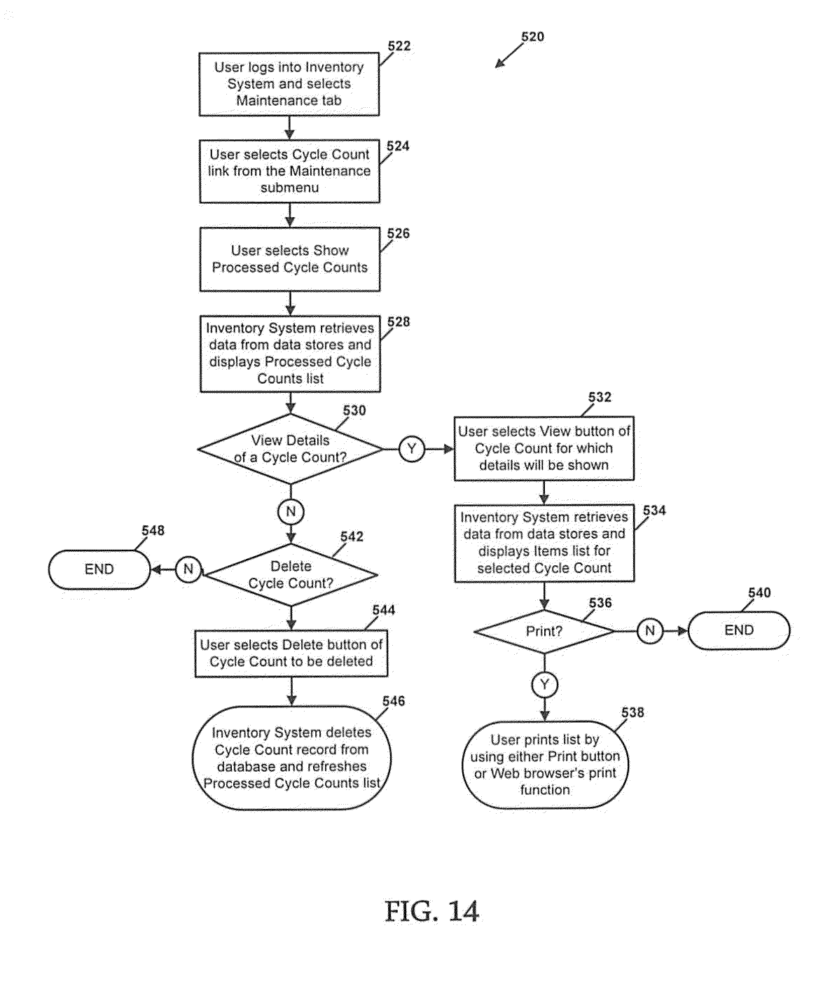

FIG. 14 depicts a process 520 which allows the customer to view a processed cycle count. To begin this process, a user logs into the inventory management application 28 running on the inventory management computer 26 via the Internet 16 and selects the Maintenance tab on the browser screen as depicted in FIG. 37 (step 522) and selects the Cycle Count link 458 in the Maintenance submenu (step 524). The user then selects to show “Processed” cycle counts from the dropdown menu 550 (step 526), at which point the inventory management application 28 retrieves cycle count data and displays this information as a Processed Cycle Counts list on the screen (step 528). The user can then choose to view details of the processed cycle count (steps 530 and 532) at which point the inventory management application 28 retrieves and displays an item list for the selected processed cycle count (step 534). The user can print the list such as by using the browser's print function (steps 536 and 538). The user may also choose to delete a processed cycle count (steps 542 and 544) at which point the inventory management application 28 deletes the selected processed cycle count and updates the processed cycle count list accordingly (step 546).

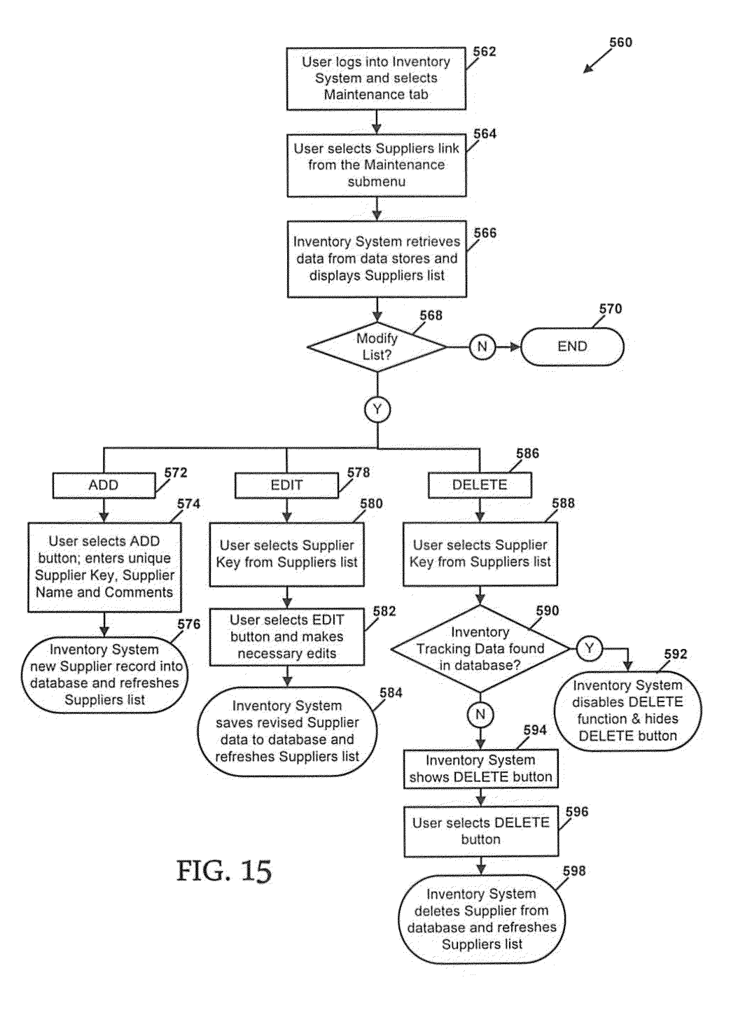

FIG. 15 depicts a process 560 for maintaining supplier information. To begin this process, a user logs into the inventory management application 28 running on the inventory management computer 26 via the Internet 16 and selects the Maintenance tab on the browser screen as depicted in FIG. 39 (step 562) and selects the Suppliers link 600 in the Maintenance submenu (step 564). The inventory management application 28 retrieves supplier data, and displays this information in a supplier list 602 on the screen (step 566). Using the options provided in the Supplier Details box 604, the user can add suppliers (steps 572-576), edit suppliers (steps 578-584), or delete suppliers (steps 586-598) as shown in FIG. 15.

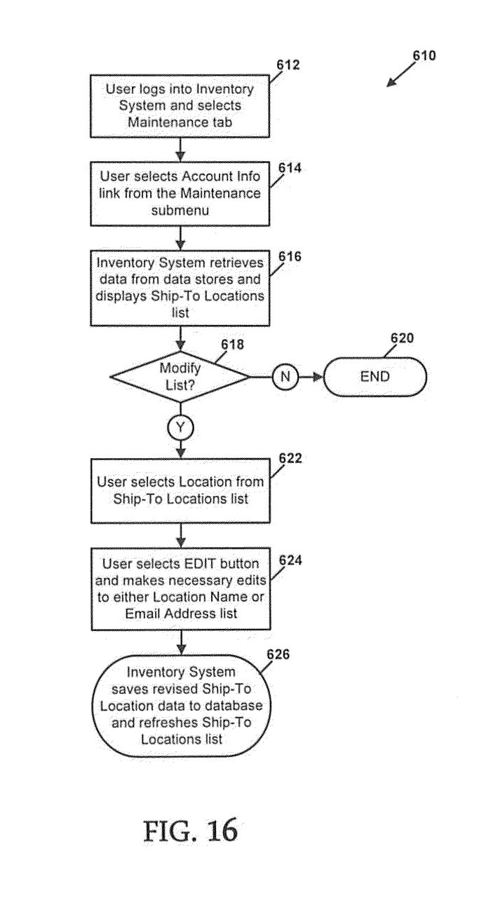

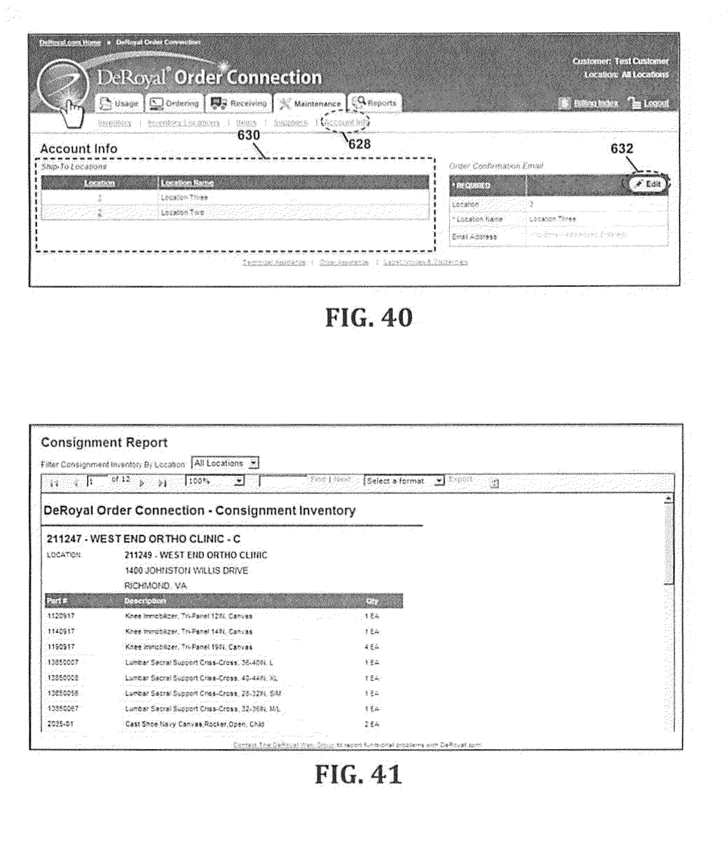

FIG. 16 depicts a process 610 for maintaining ship-to location information. To begin this process, a user logs into the inventory management application 28 running on the inventory management computer 26 via the Internet 16 and selects the Maintenance tab on the browser screen as depicted in FIG. 40 (step 612) and selects the Account Info link 628 in the Maintenance submenu (step 614). The inventory management application 28 retrieves ship-to location data, and displays this information in a Ship-To Locations list 630 on the screen (step 616). Using the Edit button 632, the user may then change the location name or the email address of the person who is to receive shipment confirmation information (steps 618-626) for the selected ship-to location.

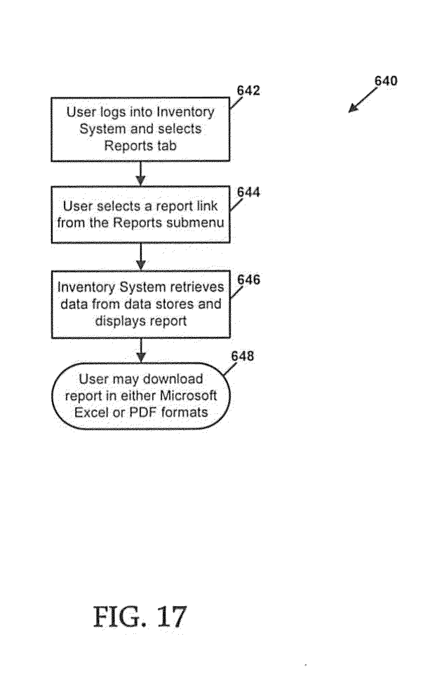

FIG. 17 depicts a process 640 for viewing and printing reports. To begin this process, a user logs into the inventory management application 28 running on the inventory management computer 26 via the Internet 16 and selects the Reports tab (step 642) and selects a report link in the Reports submenu (step 644). The inventory management application 28 retrieves the report data and displays the selected report (step 646) at which point the user may download the report in a desired format (step 648).

The foregoing description of preferred embodiments for this invention have been presented for purposes of illustration and description. They are not intended to be exhaustive or to limit the invention to the precise form disclosed. Obvious modifications or variations are possible in light of the above teachings. The embodiments are chosen and described in an effort to provide the best illustrations of the principles of the invention and its practical application, and to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. An inventory management system for managing information related to items used during a medical surgical procedure, the inventory management system comprising:
an inventory management computer for executing an inventory management application to process item usage information which includes item identification information and quantity information for each item used during performance of the medical surgical procedure, wherein the inventory management application associates the item usage information with a particular surgical case at a particular customer surgical facility, the inventory management computer in communication with a wide area network through which the inventory management application receives the item usage information from a customer computer associated with the customer surgical facility; and
an item inventory database in communication with the inventory management computer, the item inventory database for storing item inventory information.

2. The system of claim 1 wherein the inventory management application is configured to process the item usage information indicating usage of implants during performance of the medical surgical procedure.

3. The system of claim 1 wherein the inventory management application is configured to process the item usage information which further includes information indicating whether an item was implanted during the surgical procedure or was wasted.

4. The system of claim 1 wherein
the inventory management application is configured to communicate a listing of sets of items through the wide area network for display on the customer computer associated with the customer surgical facility,
the inventory management application is configured to receive a selection through the wide area network from the customer computer indicating which of the sets of items in the listing is to be associated with the particular surgical case, and
the inventory management application is configured to generate an inventory control document for the selected set of items on which a user at the customer surgical facility may record the item usage information.

5. The system of claim 1 wherein
the inventory management application is configured to communicate a listing of surgical cases associated with the customer surgical facility through the wide area network for display on the customer computer, and
the inventory management application is configured to receive a selection through the wide area network from the customer computer indicating a surgical case in the listing for which item usage is to be recorded.

6. The system of claim 5 wherein,
for the selected surgical case, the inventory management application is configured to receive usage information entered by a user on the customer computer and communicated through the wide area network identifying a set of items used during the surgical procedure, identifying each item in the set that was used during the surgical procedure, indicating a quantity of each item used, and indicating whether each item used was implanted during the surgical procedure or was wasted, and
the inventory management application is configured to update the item inventory information in the item inventory database based on the usage information received from the customer computer.

7. The system of claim 6 wherein the inventory management application is configured to receive the usage information that is manually entered using an input device connected to the customer computer.

8. The system of claim 6 wherein the inventory management application is configured to receive the usage information entered using a barcode reader connected to the customer computer.

9. The system of claim 1 further comprising:
an admissions-discharge-transfer computer system associated with the customer surgical facility which is configured to receive and process case information for the particular surgical case, wherein the admissions-discharge-transfer computer system is in communication with the inventory management computer via the wide area network; and
the inventory management application is configured to receive the case information from the admissions-discharge-transfer computer system and associate the case information with the item usage information for the particular surgical case.

10. The system of claim 1 wherein
the inventory management application is configured to communicate a listing of sets of items in inventory at the customer surgical facility through the wide area network for display on the customer computer,
the inventory management application is configured to receive a selection through the wide area network from the customer computer indicating a set of items in the listing that the user desires to replenish with items from inventory at the customer surgical facility, and
the inventory management application is configured to generate an inventory picking document for the selected set of items which the user at the customer surgical facility can use as a guide in replenishing the selected set of items with items from inventory at the customer surgical facility.

11. The system of claim 1 wherein
the item inventory database maintains a record of items in inventory at the customer surgical facility which are available to replenish sets of items depleted during a surgical procedure at the customer surgical facility,
the inventory management application is configured to communicate a listing of sets of items through the wide area network for display on the customer computer, wherein the listing indicates sets of items recorded in the item inventory database as being in inventory at the customer surgical facility,
the inventory management application is configured to receive a selection through the wide area network from the customer computer indicating a set of items that are to be transferred out of the inventory at the customer surgical facility because one or more of the items have been used to replenish a set of items depleted during a surgical procedure,
the inventory management application is configured to generate a usage list for the selected set of items which is displayed on the customer computer, wherein the user at the customer surgical facility may enter quantities of items on the usage list to be transferred from inventory at the customer facility to reflect actual usage of the items during the surgical procedure, and
the inventory management application is configured to operate on the item inventory database to adjust a record of items in inventory at the customer surgical facility to reflect actual usage as indicated by quantities of items entered by the user on the usage list.

12. The system of claim 1 further comprising a user authentication database in communication with the inventory management computer, the user authentication database for storing customer information.

13. A computer-implemented method for managing information related to implants used during a medical surgical procedure at a customer surgical facility, the method comprising:
(a) receiving implant usage information at an inventory management computer via a wide area network from a customer computer associated with the customer surgical facility, wherein the implant usage information includes implant identification information and implant quantity information for each implant used during performance of the medical surgical procedure; and
(b) associating the implant usage information with a particular surgical case at the customer surgical facility in an implant inventory database.

14. The method of claim 13 wherein step (a) includes receiving implant usage information which includes information indicating whether an implant was implanted during the surgical procedure or was wasted.

15. The method of claim 13 further comprising:
(c) communicating a listing of sets of implants from the inventory management computer through the wide area network for display on the customer computer associated with the customer surgical facility;
(d) receiving a selection at the inventory management computer communicated through the wide area network from the customer computer, the selection indicating which of the sets of implants in the listing is to be associated with the particular surgical case; and
(e) generating an inventory control document for the selected set of implants on which a user at the customer surgical facility may record the implant usage information.

16. The method of claim 13 further comprising:
(c) communicating a listing of surgical cases associated with the customer surgical facility from the inventory management computer through the wide area network for display on the customer computer;
(d) receiving a selection at the inventory management computer communicated through the wide area network from the customer computer indicating a surgical case in the listing for which implant usage is to be recorded;
(e) for the selected surgical case, receiving usage information at the inventory management computer entered by a user on the customer computer and communicated through the wide area network, the usage information identifying a set of implants used during the surgical procedure, identifying each implant in the set that was used during the surgical procedure, indicating a quantity of each implant used, and indicating whether each implant used was implanted during the surgical procedure or was wasted; and
(f) updating the implant inventory information in the implant inventory database based on the usage information received from the customer computer.

17. The system of claim 16 further comprising manually entering the usage information at the customer computer using an input device connected to the customer computer.

18. The system of claim 16 further comprising entering the usage information at the customer computer using a barcode reader connected to the customer computer.

19. The method of claim 13 further comprising:
(c) receiving and processing case information for the particular surgical case at an admissions-discharge-transfer computer system associated with the customer surgical facility, wherein the admissions-discharge-transfer computer system is in communication with the inventory management computer via the wide area network;
(d) receiving the case information from the admissions-discharge-transfer computer system at the inventory management computer; and
(e) associating the case information with the implant usage information for the particular surgical case.

20. The method of claim 13 further comprising:
(c) communicating a listing of sets of implants in inventory at the customer surgical facility from the inventory management computer through the wide area network for display on the customer computer;
(d) receiving a selection at the inventory management computer communicated through the wide area network from the customer computer, the selection indicating a set of implants in the listing that the user desires to replenish with implants from inventory at the customer surgical facility; and
(e) generating an inventory picking document for the selected set of implants which the user at the customer surgical facility can use as a guide in replenishing the selected set of implants with implants from inventory at the customer surgical facility.

21. The method of claim 13 further comprising:
(c) maintaining in the implant inventory database a record of implants in inventory at the customer surgical facility which are available to replenish sets of implants depleted during a surgical procedure at the customer surgical facility;
(d) communicating a listing of sets of implants from the inventory management computer through the wide area network for display on the customer computer, wherein the listing indicates sets of implants recorded in the implant inventory database as being in inventory at the customer surgical facility;
(e) receiving a selection at the inventory management computer communicated through the wide area network from the customer computer indicating a set of implants that are to be transferred out of the inventory at the customer surgical facility because one or more of the implants have been used to replenish a set of implants depleted during a surgical procedure;
(f) generating a usage list for the selected set of implants which is displayed on the customer computer, wherein the user at the customer surgical facility may enter quantities of implants on the usage list to be transferred from inventory at the customer facility to reflect actual usage of the implants during the surgical procedure; and
(g) operating on the implants inventory database to adjust a record of implants in inventory at the customer surgical facility to reflect actual usage as indicated by quantities of implants entered by the user on the usage list.
